(19) United States
(12) Patent Application Publication
(10) Pub. No.: US 2010/0266712 A1
(43) Pub. Date: Oct. 21, 2010

(54) Prevention of Acute Kidney Injury

(75) Inventors: Rinaldo Bellomo, Ivanhoe (AU); Michael Hasse, Potsdam (DE)

Correspondence Address: DLA PIPER LLP (US), 4365 EXECUTIVE DRIVE, SUITE 1100, SAN DIEGO, CA, 92121-2133, US

(73) Assignee: Austin Health, Heidelberg (AU)

(21) Appl. No.: 12/278,963

(22) PCT Filed: May 24, 2007
(86) PCT No.: PCT/AU07/00717

§ 371 Date: Jun. 25, 2010

(51) Int. Cl. A61K 33/00; C01D 7/00; A61P 13/12

(52) U.S. Cl. 424/717; 423/422

(57) ABSTRACT

The present invention provides a method of attenuating or preventing acute kidney injury occurring in a subject following cardiac surgery involving cardio-pulmonary bypass. The method comprises administration of at least one agent which cause alkalinization of urine to the subject at a level effective to cause alkalinization of the subject's urine. Preferably the agent which cause alkalinization of urine is selected from the group consisting of sodium bicarbonate, lactate, acetate, citrate, tromethamine and combinations thereof and is most preferably sodium bicarbonate.

### FIELD OF THE INVENTION

The present invention relates to a method of attenuating acute kidney injury following cardiac surgery. More particularly, the invention relates to the administration of sodium bicarbonate to cause alkalinization of the urine in order to prevent such injury.

### BACKGROUND TO THE INVENTION

With over one million operations a year worldwide, cardiac surgery is one of the most common major surgical procedures (Albert M A et al., 2003). Acute kidney injury (AKI) is a common post-operative complication following exposure to cardiopulmonary bypass (CPB) (Chertow G M et al., 1998). AKI requiring dialysis occurs in up to 5% of patients undergoing elective cardiac surgery. An additional 8-15% of patients have moderate AKI with an increase in serum creatinine level of greater than 1.0 mg/dL (88.4 μmol/L). Lesser degree of AKI with a greater than 25% increase in serum creatinine from baseline to post-operative peak level may affect more than 50% of patients (Stafford-Smith M et al., 2005).

Some patients are at particular risk of developing CPB-related acute renal failure such as those with an increased duration of CPB, a pre-operative serum creatinine >1.2 mg/dL, insulin dependent diabetes mellitus, age >70 years, reduced left ventricular function, valve surgery, pre-operative atrial fibrillation and vascular disease (Thakar C V et al., 2005). Interestingly, there is evidence that a longer duration of CPB is associated with an increased likelihood of and more severe AKI (Conlon P J et al., 1999).

In these patients, reducing the incidence and severity of post CPB AKI might prevent expenditure and morbidity and improve other outcomes.

AKI carries a significant cost and is a serious post-operative complication. Also, AKI leads to a significant increase in hospital expenditure especially if complicated by the need for dialysis.

Adverse outcomes of AKI after cardiac surgery include prolonged intensive care unit and hospital stay and discharge to extended-care facilities. After adjustment for co-morbidities and intra-operative variables, all degrees of AKI are associated with increased mortality (Chertow G M et al., 1998). Even minimal increments in serum creatinine are associated with an independent increase in mortality.

Multiple causes of AKI following cardiac surgery have been proposed, including peri-operative haemodynamic instability and impaired renal blood flow, ischaemia-reperfusion injury, and CPB-induced activation of inflammatory pathways and the generation of radical oxygen species (ROS). Other less common sources of renal injury include atheroembolism into the renal arteries and exogenous nephrotoxins such as nephrotoxic antibiotics, non-steroidal anti-inflammatory drugs and anaesthetics, all of which may contribute to AKI in selected patients (Stafford-Smith et al., 2005).

However, ischaemia-reperfusion injury and the generation of oxido-inflammatory stress represent two conventionally accepted major mechanisms in the pathogenesis of CPB-related AKI. The evidence supporting such mechanisms, however, is indirect and weak. In particular no randomised controlled trials seeking to prophylactically affect such pathways has yet delivered an effective therapy.

Several randomized controlled trials (RCT) have attempted to prevent or attenuate AKI, but most of these interventions have been found to be ineffective, or inconclusive and/or have only been studied in specific cardiac surgery subpopulations. For example, none of the medications which were believed to improve renal perfusion, to increase renal blood flow or to decrease cortical oxygen consumption has been consistently found to prevent or attenuate AKI including fenoldopam, dopamine, clonidine, diltiazem, nesritide, pentoxifylline and ACE inhibitors and diuretics.

CPB is involved in causing haemolysis by mechanical destruction of the erythrocytes thus generating free haemoglobin (Takami Y et al., 1996). Many sources of haemolysis contribute to increased plasma levels of free serum haemoglobin during the use of CPB and in the early post-operative period.

Shear stress on erythrocytes resulting from contact with foreign surfaces of the bypass circuit (boundary layer of oxygenator, filters, tubing), cross-sectional area, the number of circuit connectors, blood aspiration by cardiotomy suction, and the roughness of surface of the pump—all of those aggravated by high flow and high-pressure conditions—are important determinants of haemolysis.

Free haemoglobin levels of greater than 150 mg/dL, which is about 10-fold of the upper physiological range, have been observed during the use of CPB until several hours post-operatively despite the short duration of CPB (85 min). However, the detrimental effect of CPB on red cell destruction is accentuated by prolongation of CPB time. Thus, the longer the duration of CPB the more haemolysis occurs and the more free haemoglobin is likely generated. This may be of importance to the current clinical situation where complex surgery of the aortic arch and aortic valve is performed and an increasing number of cardiac surgical centres have implemented time-consuming arterial coronary revascularisation aiming to improve long-term results. Interestingly, this approach increases the duration of CPB, which, in turn, increases the likelihood of and severity of AKI.

The present inventors have found that urinary alkalinization may be protective to the kidney in high-risk patients undergoing CPB possibly by reducing CPB-associated generation and toxicity of reactive oxygen species, which, in turn may be exacerbated by hemolysis. More particularly, the inventors have found that peri-operative sodium bicarbonate infusion attenuates acute kidney injury in cardiac surgical patients receiving cardiopulmonary bypass.

### SUMMARY OF THE INVENTION

In a first aspect the present invention provides a method of attenuating or preventing acute kidney injury occurring in a subject following cardiac surgery involving cardio-pulmonary bypass, the method comprising administration of at least one agent which cause alkalinization of urine to the subject at a level effective to cause alkalinization of the subject's urine.

In a second aspect the present invention provides a method of attenuating or preventing acute kidney injury occurring in a subject following a surgical procedure wherein the procedure results in an increase in free haemoglobin levels due to haemolysis and/or the release of oxygen radicals, the method comprising administration of at least one agent which cause alkalinization of urine to the subject at a level effective to cause alkalinization of the subject's urine.

In a third aspect the present invention provides for the use of at least one agent which cause alkalinization of urine in the manufacture of a medicament for attenuating or preventing kidney injury occurring in a subject following cardiac surgery involving cardio-pulmonary bypass.

In a fourth aspect the present invention provides for the use of at least one agent which cause alkalinization of urine in the manufacture of a medicament for attenuating or preventing kidney injury occurring in a subject following a medical procedure wherein the procedure results in an increase in free haemoglobin levels due to haemolysis.

It is preferred that the agent which cause alkalinization of urine is selected from the group consisting of sodium bicarbonate, lactate, acetate, citrate, tromethamine and combinations thereof. It is most preferred that the agent is sodium bicarbonate.

### BRIEF DESCRIPTION OF THE FIGURES

FIG. 1A shows absolute changes in plasma creatinine concentration after cardiac surgery from baseline to peak value at any time within the first five post-operative days displayed as mean (SEM). Sodium bicarbonate treated patients experienced a significant attenuation in their absolute increase in plasma creatinine compared to sodium chloride patients; P=0.01 (Mann-Whitney-U test). To convert plasma creatinine in mg/dL to μmol/L, multiply by 88.4.

FIG. 1B shows absolute changes in urinary neutrophil gelatinase-associated lipocalin (NGAL) concentration after cardiac surgery from baseline to peak value at any time within the first twenty-four hours post-operatively displayed as mean (SEM). Sodium bicarbonate patients experienced a significant attenuation in their absolute increase in urinary NGAL compared to sodium chloride patients; P=0.009 (Mann-Whitney-U test).

FIG. 1C shows urine pH before (0 hours) and at 6 hours and 24 hours after commencement of sodium bicarbonate or sodium chloride infusion displayed as mean (SEM). P values for group comparisons before and at 6 hours and 24 hours: *P=0.60 and †P<0.001.

### DETAILED DESCRIPTION OF THE INVENTION

In a first aspect the present invention provides a method of attenuating or preventing acute kidney injury occurring in a subject following cardiac surgery involving cardio-pulmonary bypass, the method comprising administration of at least one agent which cause alkalinization of urine to the subject at a level effective to cause alkalinization of the subject's urine.

In a second aspect the present invention provides a method of attenuating or preventing acute kidney injury occurring in a subject following a surgical procedure wherein the procedure results in an increase in free haemoglobin levels due to haemolysis and/or the release of oxygen radicals, the method comprising administration of at least one agent which cause alkalinization of urine to the subject at a level effective to cause alkalinization of the subject's urine.

In a third aspect the present invention provides for the use of at least one agent which cause alkalinization of urine in the manufacture of a medicament for attenuating or preventing kidney injury occurring in a subject following cardiac surgery involving cardio-pulmonary bypass.

In a fourth aspect the present invention provides for the use of at least one agent which cause alkalinization of urine in the manufacture of a medicament for attenuating or preventing kidney injury occurring in a subject following a medical procedure wherein the procedure results in an increase in free haemoglobin levels due to haemolysis.

It is preferred that the agent which cause alkalinization of urine is selected from the group consisting of sodium bicarbonate, lactate, acetate, citrate, tromethamine and combinations thereof. It is most preferred that the agent is sodium bicarbonate.

The subject may be a human or non-human animal but is preferrably a human.

Typically, the medical procedure will involve cardiopulmonary bypass in conjunction with cardiac surgery.

Preferably, the sodium bicarbonate is administered to the subject from the time of induction of anaesthesia prior to cardiac surgery and continuously for twenty-four hours after cardiac surgery.

Preferably, the sodium bicarbonate is administered to the subject at a dose of about 0.45 to about 1.0 mEq/kg body weight over 1 hour followed by continuous intravenous administration of about 0.15 to about 0.5 mEq/kg/hr over 23 hours.

Preferably, the sodium bicarbonate is administered intravenously.

Suitable diluents for sodium bicarbonate will be known to persons skilled in the art of the present invention. The preferred diluent according to the invention is 5% dextrose.

By “alkalinization” it is meant that the pH of the subject's urine is higher following the administration of the sodium bicarbonate relative to the pH of the subject's urine prior to the administration of the sodium bicarbonate.

Preferably, the pH of the subject's urine following administration of the agent, preferably sodium bicarbonate, is greater than about 6, more preferably greater than about 6.5.

Methods of determining whether kidney injury has occurred in a subject will be known to persons skilled in the art of the present invention. Serum creatinine levels and urinary output are the most commonly used clinical indicators of renal function. Typically, these are measured for up to 120 hours post-operatively. Serum creatinine is measured in a blood sample taken from the subject. The typical reference range for women is considered to be 0.5 to 1.0 mg/dL (about 45-90 μmol/l) and for men 0.7 to 1.2 mg/dL (60-110 μmol/l).

Alternatively, creatinine clearance may be measured which is a more sensitive indicator of renal dysfunction. Typically, this is measured over the first 24 hours post-operatively. Methods for measuring creatinine clearance in the urine will be known to persons skilled in the art of the invention.

In a preferred embodiment of the invention, diagnosis of significant acute kidney injury is defined as an increase in serum creatinine concentration greater than 0.5 mg/dL (44 μmol/L) or greater than 25% from baseline to peak value at any time within the first five post-operative days.

Other indicators, such as cystatin C (cystatin 3) may be measured as an indicator of kidney function. Cystatin C is a serum protein used mainly as a measure of glomerular filtration rate.

Attenuation of acute kidney failure may be determined according to any of the methods described above. Preferably, the attenuation of acute kidney failure is determined by changes in the plasma creatine concentration within the first five post-operative days and/or changes in the urinary neutrophil gelatinase-associated lipocalin (NGAL) concentration within the first 24 hours post-operatively. NGAL is a biomarker of tubular injury. Further additional determinants include changes in the acid-base status of the subject's urine, need for renal replacement therapy, length of intensive care unit and hospital stay.

Preferably, the subject is one who is at particular risk of renal injury. More preferably, the subject has at least one of the following indices (a) age >70 years; (b) preoperative creatinine >0.12; (c) New York Heart Association symptom severity class 3 or 4: (d) valve surgery; (e) insulin dependent diabetes mellitus; or (f) redo surgery.

In a preferred embodiment, the assessment of renal function will be determined from a blood sample obtained from the subject. Preferably, the blood sample is taken from the arterial cannula routinely inserted in all subjects undergoing cardiac surgery. Preferably samples of blood are taken immediately after the induction of anaesthesia, on arrival in the intensive care unit following surgery, and at about 6, 12 and 24 hours post-operatively and every day thereafter.

In order that the nature of the present invention may be more clearly understood, preferred forms thereof will now be described with reference to the following non-limiting examples.

### Example 1

A double-blind, randomised, placebo controlled trial of the effect of sodium bicarbonate on postoperative renal function and oxidative stress in subjects undergoing elective cardiopulmonary bypass surgery was conducted.

### Materials and Methods

### Patients

The Human Research Ethics Committee of the Austin Hospital approved the trial and the Therapeutic Goods Administration of the Australian Government, Department of Health and Ageing approved the use of sodium bicarbonate for this indication. Written informed consent was obtained from each patient. Patients were identified in surgical pre-admission clinics and on hospital wards. Patients who were enrolled were at high risk of post-operative AKI and were scheduled for elective cardiac surgery necessitating the use of CPB at a university tertiary referral hospital (Austin Hospital) and a co-located large private hospital (Warringal Hospital). Those considered at high risk for post-operative AKI, defined as fulfilling one or more pre-defined inclusion criteria and no exclusion criteria, were considered eligible. Patients at particular risk of post-bypass renal injury were defined as having at least one of the following: (a) age >70; (b) preoperative creatinine >0.12; (c) New York Heart Association symptom severity class 3 or 4; (d) valve surgery; (e) insulin dependent diabetes mellitus; or (f) redo surgery.

### Patients

Body weight adjusted dose of study medication was achieved by infusion of 154 mEq/L of sodium bicarbonate (Pfizer, Bentley, WA, Australia) or sodium chloride (Astra Zeneca, North Ryde, NSW, Australia) diluted in 5% dextrose (Baxter, Sydney, NSW, Australia) and H₂O at an infusion rate of 3 mL kg⁻¹ h⁻¹ for 1 hour immediately after the induction of anesthesia, prior to the first surgical incision followed by continuous infusion at an infusion rate of 1 mL kg⁻¹ n⁻¹ over 23 hours. Sodium bicarbonate or sodium chloride was administered at a dose of 0.45 mEq/kg body weight over 1 hour followed by continuous intravenous infusion of 0.15 mEq/kg/hr over 23 hours (total dose of 4 mEq/kg over 24 hours).

### Study Design

The hospital pharmacy clinical trials coordinator used a Microsoft Excel-based (Microsoft Corp. Redmond, Wash., USA) random number generator to create the randomization list using a permuted block strategy with blocks of six. Infusion bags were each delivered in separate shrink-wrapped black plastic bags that were identical in appearance. Allocation concealment to patients, anesthesiologists, cardiac surgeons, intensive care specialists, bedside nurses and investigators (multiple blind) was ensured by central randomization through the Department of Pharmacy at the Austin Hospital. Treatment allocation was only revealed after data analysis had been performed.

### Outcome Measures

The primary study outcome measure was the number of patients who developed post-operative acute kidney injury. This was defined as an increase in plasma creatinine concentration greater than 0.5 mg/dL (44 μmol/L) or greater than 25% from baseline to peak value at any time within the first five post-operative days. This definition is identical to that used in a recent large randomized controlled trial of cardiac surgery-associated AKI.

The renal secondary outcomes for this study were changes in plasma creatinine concentration within the first five post-operative days and in urinary neutrophil gelatinase-associated lipocalin (NGAL) concentration within the first 24 hours post-operatively. Creatinine was sampled daily and NGAL was sampled prior to induction of anesthesia, at 6 hours and at 24 hours after commencement of CPB. Creatinine was measured using the modified Jaffé method and NGAL as recently described (Mishra et al, 2005).

Additional secondary outcomes included changes in acid-base status, clinical outcomes such as need for renal replacement therapy, duration of ventilation, length of intensive care unit and hospital stay, and adverse events.

Specific adverse events targeted for detection included the incidence of hypernatremia ([Na⁺]>150 mEq/L), hypokalemia ([K⁺]<3.5 mEq/L), alkalemia (pH>7.50), post-operative atrial fibrillation and other post-operative arrhythmias (supraventricular arrhythmias, ventricular tachycardia and ventricular fibrillation) during study treatment.

### Statistical Analysis

Using data available from the institutional Cardiac Surgery database and from the literature it was estimated that, in the control group, 50% of the high-risk patients would develop an increase in plasma creatinine greater than 0.5 mg/dL (44 μmol/L) or greater than 25% from baseline. Given a minimal clinically important reduction in the incidence of acute kidney injury (as defined) from 50% to 30%, it was calculated that 100 patients would be needed to have a 90% power to detect a difference between the control and the intervention group at an alpha of 0.05.

All data were analyzed according to the intention-to-treat principle. Continuous data were tested for normal distribution using histograms. Between-group comparisons for continuous data were performed with the use of the t test or the Mann-Whitney U test and for categorical data with the use of Fisher exact test or chi-square test where appropriate. All tests were 2-tailed and a P value <0.05 was taken to indicate statistical significance. Values were reported as means with standard error of the mean (SEM) and mean difference or odds ratio (OR) with 95% confidence interval (95% CI) estimate as appropriate.

### Results

Between June 2006 and December 2006, 100 patients were randomized to receive intravenous sodium bicarbonate (N=50) or sodium chloride (N=50). One sodium bicarbonate patient received nine hours of study treatment until the infusion bag was damaged by accident. One patient in the control group received seven hours of study treatment until the treating intensive care specialist stopped the infusion due to a cardiac arrest.

Patient distribution to treatment groups was well balanced according to study hospital (P=0.55). There were no significant differences between the groups in baseline characteristics including plasma creatinine and urinary NGAL concentrations (Table 1).

Most patients had elective cardiac surgery (Table 2). Treatment groups did not differ significantly in duration of CPB as well as medical interventions during and after cardiac surgery (Table 2). Mean dose of sodium bicarbonate was 307±8 mEq and mean dose of sodium chloride was 309±10 mEq (P=0.89).

### Outcomes

Fewer patients in the sodium bicarbonate group (16/50) developed post-operative AKI compared to the control group (26/50) (OR 0.43 [95% CI 0.19-0.98]), (P=0.04).

There was also a trend toward fewer patients with a plasma creatinine increase greater than 50% from baseline to peak value (8/50 vs. 15/50; OR 0.44 [95% CI 0.17-1.17] P=0.10) in patients treated with sodium bicarbonate.

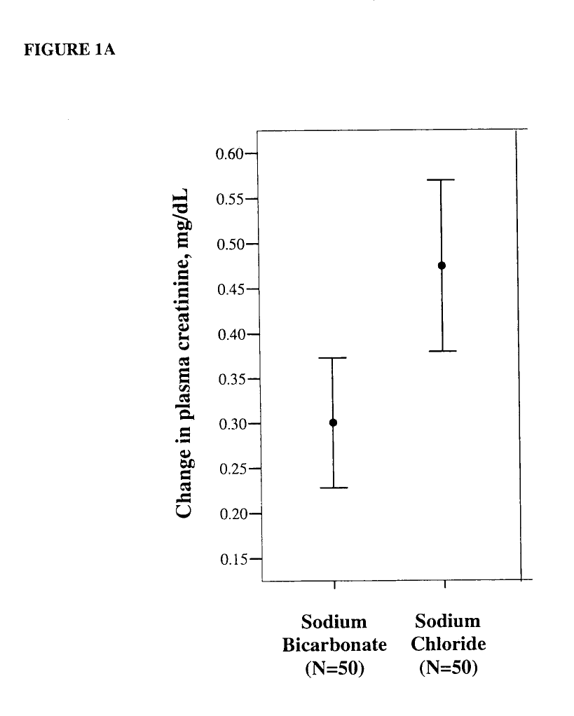

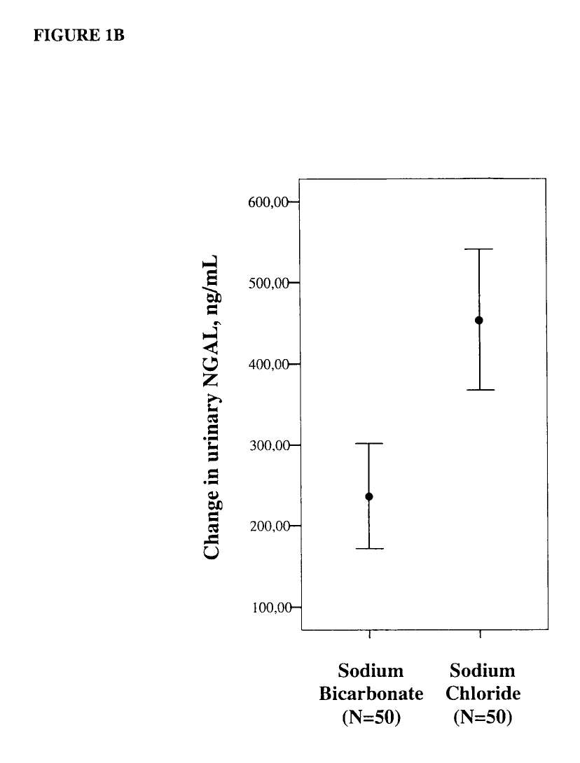

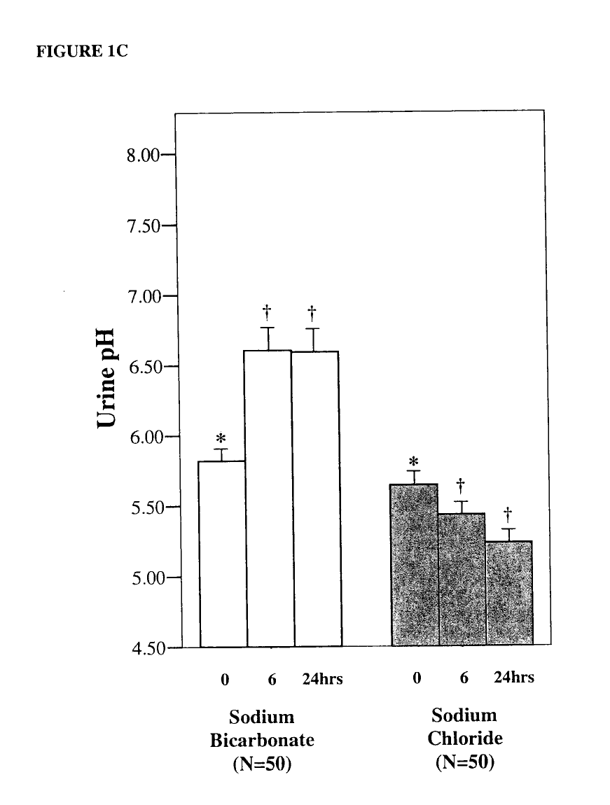

The absolute increase in plasma creatinine concentration was less in sodium bicarbonate patients (P=0.01) (FIG. 1A). The relative increase in plasma creatinine was 28.5±6.5% in sodium bicarbonate patients and 46.7±8.0% in sodium chloride patients (P=0.01).

Also, the increase in urinary NGAL concentration was attenuated in sodium bicarbonate patients (P=0.009) (FIG. 1B).

There were marked group differences in plasma bicarbonate concentration, base excess, and pH from baseline to 24 hours (all P<0.001) (Table 3). Sodium bicarbonate infusion induced urinary alkalinization after 6 and 24 hours after commencement of study drug infusion, whereas urine pH level decreased in control patients (P<0.001) (FIG. 1C).

No differences were found in the requirement for renal replacement therapy, duration of mechanical ventilation and hospital mortality (Table 3). Length of intensive care unit and hospital stay was similar. One patient in each study group died from treatment-resistant cardiogenic shock within the same hospital admission, both outside the study treatment period.

### Safety

No patient developed a plasma sodium concentration >150 mEq/L. One patient in the control group had a pH level below 7.30 (P>0.99) and three sodium bicarbonate patients had a pH level greater than 7.50 (P=0.24). More sodium bicarbonate patients had a plasma potassium <3.5 mEq/L compared to control (5/50 vs. 1/50, P=0.20). During treatment, there was no difference in the incidence of new-onset atrial fibrillation (5/50 vs. 4/50, P>0.99). One sodium bicarbonate and one patient in the control group had an episode of supraventricular tachycardia. Another patient in the control group had ventricular fibrillation during study drug infusion, which was initially successfully treated. The latter patient, however, died as described above.

### Discussion

A multiple-blind, randomized controlled clinical trial was conducted to investigate whether sodium bicarbonate infusion with pre-operative intravenous loading to achieve urinary alkalinization could attenuate CPB-related acute kidney injury in high-risk cardiac surgical patients. It was found that sodium bicarbonate treatment successfully alkalinized both blood and urine. Sodium bicarbonate infusion was associated with an absolute risk reduction for AKI of 20% (95% CI 1.1-39.0%) and with significant attenuation in urinary NGAL increase.

Previous double-blind randomized controlled trials prophylactically attempting to prevent or attenuate post cardiac surgery AKI targeting oxidative stress, renal ATP consumption and improvement of peri-operative hemodynamic stability have been found to be ineffective, inconclusive or studied in small and highly specific cardiac surgical subpopulations.

The simultaneous alkalinizing effect and apparent renal protection achieved with intravenous sodium bicarbonate is consistent with the biologic rationale for the trial. According to this rationale, AKI after cardiopulmonary bypass might represent a combination of tubular injury induced by reactive oxygen species and free hemoglobin release. For example, experimental studies in animals show that sodium bicarbonate protects from oxidant injury by slowing pH-dependent Haber-Weiss free radical production. It also directly scavenges peroxynitrite and other reactive species generated from nitric oxide. Urinary alkalinization with intravenous sodium bicarbonate has also been found to attenuate AKI in patients undergoing infusion of contrast media, another condition where free oxygen radical generation may be involved.

In addition, urinary alkalinization with sodium bicarbonate might have protected from free hemoglobin-mediated renal injury. Hemoglobin infusion causes acute renal failure and urinary alkalinization or hemoglobin blockade with haptoglobin attenuates renal injury. Furthermore, aciduria converts hemoglobin to methemoglobin, which precipitates, forms distal casts and induces AKI. Animal experiments show that red blood cell hemolysate is a potent mitogen for renal tubular epithelial cells, that free ferrous ions causes hydroxyl radical formation and lipid peroxidation during reperfusion of ischemic kidneys and that free-radical production catalyzed by free ferrous ions is most active at acid pH. In contrast, at neutral or alkaline pH induced by sodium bicarbonate, more free ferric ions precipitate as insoluble ferric hydroxides, reducing the production of injurious hydroxyl radicals.

The inventors have discovered, for the first time, that intravenous sodium bicarbonate attenuates post-operative AKI in cardiac surgical patients and that this treatment appears safe. In sodium bicarbonate patients, two independent surrogate parameters of renal function indicated an attenuation of renal injury. Urinary NGAL represents a sensitive, specific, and highly predictive early biomarker for AKI after cardiac surgery. The appearance of NGAL in the urine is related to the dose and duration of renal injury and precedes the appearance of other urinary markers.

Patients with a mix of cardiac surgery procedures were enrolled. Thus, these results may have implications for the majority of cardiac surgical patients. Sodium bicarbonate is relatively inexpensive, and simple to administer.

Both groups were well balanced with respect to pre-operative and intra-operative characteristics and risk factors for AKI. The internal validity of the results was strengthened by multiple blinding (patients, clinicians, data collectors and data analysts) and central randomization. Sodium bicarbonate was infused as a loading dose followed by a maintenance infusion immediately before, during and for twenty-three hours after the commencement of the CPB in order to deliver sufficient concentrations through the period of maximal risk.

Pre-existing renal impairment was not present in all patients to identify a very high-risk cohort. On the other hand, an overall high incidence of post-operative AKI was observed within the first five post-operative days. Thus, a cohort of cardiac surgical patients at high risk of AKI were correctly identified. Plasma creatinine was used as the primary outcome measure of the study. Plasma creatinine is the marker used in daily practice to guide clinical decisions worldwide and has served as surrogate parameter of renal function in previous large randomized controlled trials. More importantly, even a minimal increase of plasma creatinine is associated with elevated morbidity and mortality in cardiac surgery patients. Furthermore, the beneficial effect of bicarbonate in these patients was similar in magnitude to that reported for NAC prophylaxis in high-risk patients receiving radio-contrast media. Finally, no biochemical interference of pH changes on creatinine or NGAL measurement has been reported.

It is theoretically possible that sodium bicarbonate was not protective but rather that sodium chloride was injurious. However, prophylactic hydration with sodium chloride infusion has been shown to protect the kidney from radio-contrast nephropathy and has been used for resuscitation in innumerable patients over decades without reports of adverse renal effects.

In conclusion, the present inventors have found sodium bicarbonate to be efficacious, safe, inexpensive and easy to administer.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

TABLE 1
Pre-operative Characteristics in Patients Receiving
either Sodium Bicarbonate or Sodium Chloride infusion

| | Sodium | | Sodium | |
|---|---|---|---|---|
| | Bicarbonate | | Chloride | |
| Characteristics | (N = 50) | | (N = 50) | |
| Demographic Data | | | | |
| Age, yr, mean (SEM) | 71.5 | (1.3) | 70.6 | (1.4) |
| Male, no. (%) | 30 | (60) | 33 | (66) |
| Weight, kg, mean (SEM) | 77.7 | (2.0) | 78.2 | (2.4) |
| Inclusion Criteria | | | | |
| Age >70 yr, no. (%) | 32 | (64) | 31 | (62) |
| Undergoing valve surgery, | 35 | (70) | 38 | (76) |
| no. (%) | | | | |
| Left ventricular dysfunction, | 13 | (26) | 10 | (20) |
| no. (%)* | | | | |
| Plasma creatinine >1.4 mg/dL, | 3 | (6) | 5 | (10) |
| no. (%)† | | | | |
| Insulin dependent | 5 | (10) | 2 | (4) |
| diabetes mellitus, no. (%) | | | | |
| Redo cardiac surgery, no. (%) | 5 | (10) | 4 | (8) |
| Comorbidities | | | | |
| Plasma creatinine, | 1.04 | (0.04) | 1.01 | (0.04) |
| mg/dL, mean (SEM)† | | | | |
| Estimated glomerular filtration | 72.3 | (2.8) | 75.4 | (2.8) |
| rate, mL/min, mean (SEM)‡ | | | | |
| Neutrophil gelatinase-associated | 17.8 | (3.2) | 13.0 | (3.2) |
| lipocalin, ng/mL, mean (SEM) | | | | |
| Arterial hypertension, no. (%) | 46 | (92) | 42 | (84) |
| Hypercholesterolemia, no. (%) | 31 | (62) | 29 | (58) |
| Atrial fibrillation, no. (%) | 13 | (26) | 13 | (26) |
| Recent myocardial infarction, no. (%) | 8 | (16) | 9 | (18) |
| Chronic obstructive pulmonary | 6 | (12) | 8 | (16) |
| disease, no. (%) | | | | |
| Vascular disease, no. (%)∫ | 10 | (20) | 6 | (12) |
| Euro Score, points, mean (SEM) | 6.7 | (0.4) | 6.0 | (0.3) |
| Medication | | | | |
| Beta-receptor blockers, no. (%) | 27 | (54) | 19 | (38) |
| Calcium channel blockers, no. (%) | 14 | (28) | 9 | (18) |
| ACE inhibitors or angiotensin | 40 | (80) | 32 | (64) |
| blockers, no. (%) | | | | |
| Platelet inhibitors, no. (%) | 34 | (68) | 28 | (56) |
| Statins, no. (%) | 28 | (56) | 21 | (42) |

*left ventricular dysfunction as defined by EuroScore definition (Nashef et al., 1999);
†to convert plasma creatinine in mg/dL to μmol/L, multiply by 88.4;
‡Levey et al., (2000);
∫vascular disease summarizes peripheral vascular disease and carotid artery disease.

TABLE 2
Intra-operative Characteristics and Intra- and Post-
operative Interventions in Patients Receiving either
Sodium Bicarbonate or Sodium Chloride infusion

| | Sodium | | Sodium | |
|---|---|---|---|---|
| | Bicarbonate | | Chloride | |
| Characteristics | (N = 50) | | (N = 50) | |
| Cardiopulmonary bypass | 132.2 | (8.8) | 148.6 | (9.8) |
| time, min, mean (SEM) | | | | |
| Aortic cross-clamp time, | 105.3 | (7.0) | 113.3 | (7.9) |
| min, mean (SEM) | | | | |
| Valve Surgery | | | | |
| Valve surgery only, no. (%) | 25 | (50) | 25 | (50) |
| Aortic valve replacement, no. (%) | 14 | (28) | 12 | (24) |
| Mitral valve replacement, no. (%) | 3 | (6) | 2 | (4) |
| Mitral valve repair, no. (%) | 4 | (8) | 6 | (12) |
| Double valve surgery, no. (%) | 4 | (8) | 5 | (10) |
| Coronary Artery Bypass | | | | |
| Grafting Surgery | | | | |
| Coronary artery bypass | 15 | (30.0) | 12 | (24.0) |
| grafting surgery only, no. (%) | | | | |
| Average of vessels bypassed, | 2.6 | (0.03) | 3.0 | (0.04) |
| mean (SEM) | | | | |
| Other Cardiac Surgery | | | | |
| Concomitant valvular and coronary | 7 | (14.0) | 8 | (16.0) |
| artery bypass graft surgery, no. (%) | | | | |
| Complex cardiac surgery, no. (%) | 3 | (6.0) | 5 | (10.0) |
| Hemodynamic and Fluid Management* | | | | |
| Cardiac index, L/m² | | | | |
| lowest, mean (SEM) | 2.1 | (0.06) | 2.2 | (0.07) |
| mean, mean (SEM) | 2.8 | (0.06) | 2.9 | (0.07) |
| Mean arterial pressure, mmHg | | | | |
| lowest, mean (SEM) | 65.4 | (1.1) | 63.4 | (1.0) |
| mean, mean (SEM) | 81.3 | (1.1) | 79.1 | (1.1) |
| Intravenous fluids, mL, mean (SEM) | 5893 | (241) | 5404 | (206) |

*Within the first 48 hours post-operatively; SEM, standard error of the mean.

TABLE 3
Acid Base Status and Hospital Outcomes in Patients Receiving
either Sodium Bicarbonate or Sodium Chloride infusion

| | Sodium | Sodium | | | | |
|---|---|---|---|---|---|---|
| | Bicarbonate | Chloride | | | Mean Difference | |
| Outcomes | (N = 50) | (N = 50) | | P value | (95% CI) | |
| Acid Base Status* | | | | | | |
| Change in plasma bicarbonate, mEq/L, mean (SEM) | 3.7 (0.5) | −1.5 | (0.3) | <0.001 | 5.2 | (4.1-6.4) |
| Change in plasma pH, mean (SEM) | 0.04 (0.01) | −0.03 | (0.01) | <0.001 | 0.07 | (0.04-0.10) |
| Change in plasma base excess, mean (SEM) | 3.8 (0.5) | −2.2 | (0.4) | <0.001 | 6.0 | (4.7-7.3) |
| Hospital Outcomes | | | | | | |
| Renal replacement therapy, no. (%) | 2 (4) | 2 | (4) | >0.99 | — | |
| Length of ventilation, min, mean (SEM) | 1657 (299) | 1756 | (368) | 0.83 | −99 | (−1040-84 1) |
| Length of stay in intensive care unit, hrs, mean (SEM) | 73.8 (14.0) | 74.8 | (13.1) | 0.96 | −1.0 | (−39.0-37.0) |
| Length of stay in hospital, days, mean (SEM) | 10.4 (1.0) | 11.2 | (1.2) | 0.60 | −0.8 | (−3.9-2.3) |

| | | | | | |
|---|---|---|---|---|---|
| Hospital deaths, no. (%) | 1 (2) | 1 | (2) | >0.99 | — |

*Changes in plasma bicarbonate, plasma pH and base excess from before to 24 hours after commencement of study infusion.
pH, potentia hydrogenii; SEM, standard error of the mean.

### REFERENCES

Albert M A, Antman E M. Preoperative Evaluation for Cardiac Surgery. In: Cohn L H, Edmunds L H Jr, eds. Cardiac Surgery in the Adult. New York: McGraw-Hill, 2003:235-48.
Conlon P J, Stafford-Smith M, White W D, et al. Acute renal failure following cardiac surgery. Nephrol Dial Transplant 1999; 14:1158-62.
Chertow G M. Independent associations between acute renal failure and mortality following cardiac surgery. Am J Med 1998; 104:343-8.
Levey A S, Greene T, Kusek J W, Beck G J, MDRD Study Group. A simplified equation to predict glomerular filtration rate from serum creatinine. J Am Soc Nephrol 2000; 11:A0828.
Mishra J, Dent C, Tarabishi R, Mitsnefes M M, Ma Q, Kelly C, Ruff S M, Zahedi K, Shao M, Bean J, Mori K, Barasch J, Devarajan P. Neutrophil gelatinase-associated lipocalin (NGAL) as a biomarker for acute renal injury after cardiac surgery. Lancet. 2005 Apr. 2-8; 365(9466):1231-8.
Nashef S A, Rogues F, Michel P, et al. European system for cardiac operative risk evaluation (EuroSCORE). Eur J Cardiothorac Surg 1999; 16:9-13.
Stafford-Smith M, Podgoreanu M, Swaminathan M, et al. Association of genetic polymorphisms with risk of renal injury after coronary bypass graft surgery. Am J Kidney Dis 2005; 45:519-30.
Thakar C V, Arrigain S. Worley S, et al. A clinical score to predict acute renal failure after cardiac surgery. J Am Soc Nephrol 2005; 16:162-8.

1. A method of attenuating or preventing acute kidney injury occurring in a subject following cardiac surgery involving cardio-pulmonary bypass, the method comprising administration of at least one agent which cause alkalinization of urine to the subject at a level effective to cause alkalinization of the subject's urine.

2. A method of attenuating or preventing acute kidney injury occurring in a subject following a surgical procedure wherein the procedure results in an increase in free haemoglobin levels due to haemolysis and/or the release of oxygen radicals, the method comprising administration of at least one agent which cause alkalinization of urine to the subject at a level effective to cause alkalinization of the subject's urine.

3. A method as claimed in claim 1 wherein the agent which cause alkalinization of urine is selected from the group consisting of sodium bicarbonate, lactate, acetate, citrate, tromethamine and combinations thereof.

4. A method as claimed in claim 3 wherein the agent is sodium bicarbonate.

5. A method as claimed in claim 4 wherein the sodium bicarbonate is administered to the subject from the time of induction of anaesthesia prior to cardiac surgery and continuously for twenty-four hours after cardiac surgery.

6. A method as claimed in claim 4 wherein the sodium bicarbonate is administered to the subject at a dose of about 0.45 to about 1.0 mEq/kg body weight over 1 hour followed by continuous intravenous administration of about 0.15 to about 0.5 mEq/kg/hr over 23 hours.

7. A method as claimed in claim 3 wherein the sodium bicarbonate is administered intravenously.

8. A method as claimed in claim 1 wherein the pH of the subject's urine following administration of the agent is greater than about 6, more preferably greater than about 6.5.

9. A method as claimed in claim 1 wherein the subject is one who is at particular risk of renal injury.

10. A method as claimed in claim 1 wherein the subject has at least one of the following indices (a) age >70 years; (b) preoperative creatinine >0.12; (c) New York Heart Association symptom severity class 3 or 4; (d) valve surgery; (e) insulin dependent diabetes mellitus; or (f) redo surgery.

11. A method as claimed in claim 1 wherein the attenuation of acute kidney failure is determined by changes in the plasma creatine concentration within the first five post-operative days and/or changes in the urinary neutrophil gelatinase-associated lipocalin (NGAL) concentration within the first 24 hours post-operatively.

12. The use of sodium bicarbonate in the manufacture of a medicament for attenuating or preventing kidney injury occurring in a subject following cardiac surgery involving cardio-pulmonary bypass.

13. The use of sodium bicarbonate in the manufacture of a medicament for attenuating or preventing kidney injury occurring in a subject following a surgical procedure wherein the procedure results in an increase in free haemoglobin levels due to haemolysis.

14. The use of at least one agent which cause alkalinization of urine in the manufacture of a medicament for attenuating or preventing kidney injury occurring in a subject following cardiac surgery involving cardio-pulmonary bypass.

15. The use of at least one agent which cause alkalinization of urine in the manufacture of a medicament for attenuating or preventing kidney injury occurring in a subject following a medical procedure wherein the procedure results in an increase in free haemoglobin levels due to haemolysis.

16. The use as claimed in claim 14 wherein the agent which cause alkalinization of urine is selected from the group consisting of sodium bicarbonate, lactate, acetate, citrate, tromethamine and combinations thereof.

17. The use as claimed in claim 16 wherein the agent is sodium bicarbonate.
